(19) United States
(12) Patent Application Publication
(10) Pub. No.: US 2006/0008794 A1
(43) Pub. Date: Jan. 12, 2006

(54) HOX code functions as a growth synchronizing moving reference frame

(76) Inventors: Craig S. Rhodes, Silver Spring, MD (US); Curtis A. Rhodes, Irmo, SC (US)

Correspondence Address: Craig S Rhodes, 9121 Walden Rd, Silver Spring, MD, 20901, US

(21) Appl. No.: 10/883,812

(22) Filed: Jul. 6, 2004

(51) Int. Cl. C12Q 1/00; C12N 15/85

(52) U.S. Cl. 435/4; 435/455

(57) ABSTRACT

The determination of position during metazoan growth appears complex, in part, because we observe that growth does not occur at a constant rate, as temperatures fluctuate, so do the growth rates. One positioning method, a moving reference frame (MRF), is used in engineering, and navigation, to assign positions when velocity and changes in velocity are considerations. The use of a biological MRF employing Hox proteins to control position of cells along an axis is suggested, by the temporal-spatial expression patterns of the Hox proteins,[1, 2] the affects of Hox proteins on cell divisions. [3, 4] A MRF simultaneously controlling both gene transcription and cell divisions when coupled with asymmetric cell division seems sufficient to effectively determine exact cellular position along an axis of growth and account for many described homeotic morphological variations.[5]

### FEDERAL RESEARCH STATEMENT

[Some of the data necessary for the understanding of the HOX code may have been observed at a government laboratory. The US government may have certain rights to certain portions of this patent.]

### BACKGROUND OF INVENTION

There exists fundamental rules of physics that govern the relations of all events occurring in space and time that also must apply to the morphology of all metazoans. Spatial temporal events in one body of reference can sometimes be converted to another body of reference using transformations, such as Galilean or Lorentz transformations. Spatial transformations, termed homeosis also occur during the morphological formation of animals, these homeotic transformations have understandable causes.

Fundamental to understandings of space and time are bodies of reference, references to which position and movement, or lack of movement can be related. Galileo space-time concepts were directed by an understanding of the Copemican heliocentric solar system in which the Earth was revolving around the Sun, while rotating about its own axis and yet we perceive no effects of this motion on the predictive outcome of events involving space and time within our body of reference, the Earth. Galileo stated “Experiments on the Earth are indifferently adapted to an Earth in motion or at rest . . . . It is has been clearly explained that such motion that is common to us and to the moving bodies is as it did not exist, whatever moves, moves with respect to something that is motionless”, these are concepts of Galilean relativity.

Correct spatial-temporal formation of structures composing multi-cellular animals, termed metazoans, presents unique problems of space and time. How do organisms produce spatially related structures generation after generation, at different geological or absolute times?

A growth synchronizing moving reference seems sufficient to replicate biological morphological structures at any geological or absolute time while maintaining the fundamental rules of physics.

### SUMMARY OF INVENTION

We describe theoretical requirements for the determination of cellular positions and the involvement of position determination in cancer and neoplasias in metazoans. If position is not maintained as a direct relationship to cell division and/or gene expression, then time a variable will appear in the independent variable of a theoretical equation (governing equation(s)) that describe the growth and the various cellular positions attained during that growth.

### BRIEF DESCRIPTION OF DRAWINGS

FIG. 1. Geometric representation of axial growth and the determination of distance. A. Diagrammatic growth along an axis and the relationship between distance s and time t. B. A position versus time curve. Note the effect of moving the coordinate frame with time (dotted vertical coordinate line=t₀+Δt (change in time), this would occur if traveling in register with growth, a PZ,[12] or when using a MRF, time is removed from the independent variable of governing equations that would describe changes with displacement, or growth. C. Gradient model showing the relationship between morphogen concentration and position.[26] Note however during the gradient position determinations the exact same relationships must occur regardless of changes in gene expression or divisions occur. The position versus time graph and gradient model assessment suggests that unless position x, y, z, is always maintained as some related value to growth (either a direct function of cell division and/or gene expression) time will appear in the independent variable of a theoretical description or governing equation describing that growth. D. A moving reference frame model for axis formation. A series of three Hox genes along the chromosome and co-linear expression domains (dotted line with arrow) along the axis are diagrammed. Final allotted growth or s along the morphogenetic field, remains initially closely related to Hox values. An observer traveling in register with the moving origin (the internal reference frame) would not notice any positional changes due to growth.

FIG. 2. Predicted phenotypes if the initial position, i.e., x, y, z, determined during extension of the growth axis was required before the assignment of subordinate events. A. A null mutation extends the t₀ domain into the t2 as an earlier morphological domain occupies both the normal domain, as well as an adjacent later growth time morphological domain (from behind moving forward, a forward homeosis[5]). B. A gain-of-function mutation shifts a t3 domain into a t2 domain and alters an early time anatomical region to resemble what should have appeared only as a later time structure (from before moving backward, a backward homeosis[5]). C. Phocomelic dysmorphologies,[11] such as those as caused by thalidomide are diagrammed as a homeosis. This terminology is agreement with homeotic principles,[5] since both a reduction in meristic segments (a reduction in the number of limb parts in a series), as well as a transfer of the distal limb part to occupy a proximal domain with missing segments (a loss of normal limb ordinal relationships and a homeosis) can occur due to thalidomide-induced congenital abnormalities. Slight variations in MRF positions along two different axes, such as a left versus right arm, would be expected during embryonic growth, and could explain the asymmetric affects of thalidomide on limbs.[27] D. Alteration of cis elements responding to the HOX code would alter position of body features or organs, for example supernumary breasts, to include different segment positioning. Controlling cell divisions and gene expression in synchrony would lead to alterations in positioning of structures from the beginning of each segment as indicated.

### DETAILED DESCRIPTION

The Hox proteins, a large family of transcription regulatory factors that contain a conserved DNA-binding domain, the homeobox, are involved in determining the position and appearance of morphological structures in all metazoans. Complex and dramatic phenotypic changes are produced by alterations in Hox proteins, however, these phenotypes might be due to a pivotal role of the Hox proteins in the first steps of a series of developmental steps, [6, 7] each subsequent step of which is dependent upon the previous. Hox ge nes are arranged along chromosomes in groups, and during the formation of a morphological axis, such as the major body axis during gastrulation, or the limb axis during limb bud outgrowth, a series of Hox are expressed in the same sequential order as positioned along a chromosome, termed co linearity.[1, 2] First described during the cataloging of discontinuous morphological variations, the positions of morphological structures change, but frequently change in a particular manner in relationship to their positions in related body plans; predominately, either moving forward along an axis, “from behind traveling forward”, a forward homeosis, or backward along an axis “from before traveling backward” a backward homeosis.[5] Mutations in gene complexes controlling segmentation, now identified as invertebrate Hox genes, [8] and alterations in the expression of vertebrate Hox genes,[9, 10] can alter the positions of morphological domains along axes, by moving domains forward or backward along an axis.

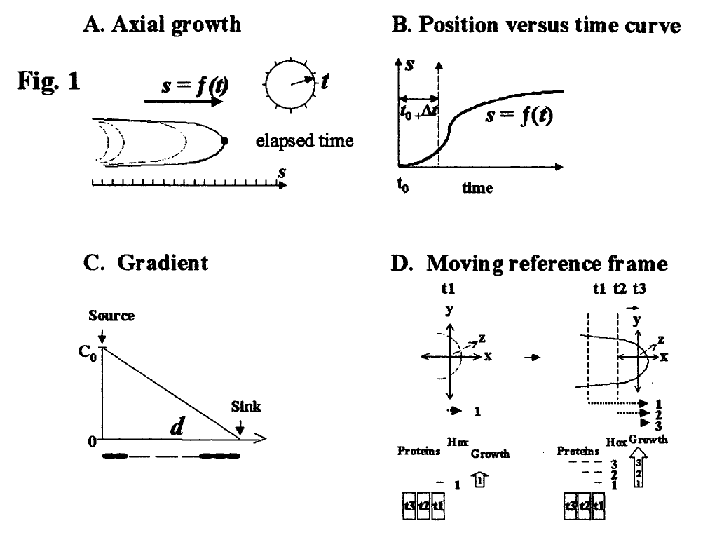

Growth of metazoans along an axis can be represented geometrically. Suppose that growth is occurring along a straight line, and a coordinate line is introduced in the direction of growth along the axis (FIG. 1a). Imagine a dock to keep track of the elapsed time, starting with t=0 initially, and after seconds have elapsed the growth tip is s units from the origin. Because s changes with t, the position coordinate s is some function of t, equation (1.1). The graph of this function with the t axis horizontal and s axis vertical is a position versus time curve. (FIG. 1b) Since reports have not indicated that cell sizes are varying along an axis, we can represent distance along a morphological axis by equation (1.2). Distance s is then is an unknown function of both total number of cell divisions and a sum quantity of gene expression affecting the extracellular spacing that has occurred.
s=f(t) (1.1)
s=f(cell divisions+gene expression) (1.2)

Principally, two types of models either gradient or progressive ordering models (proximal-distal ordering[11] and progress zone PZ[12]), have been proposed for determining cellular positions along a morphological axis and have been reviewed recently,[13-15] so only a brief description with a focus on rate change effects is presented. The gradient model suggests that after some unknown amount of growth or distance, s, position is determined by a direct relationship to the concentration of a substance the morphogen, termed an isomorphic position determination. (FIG. 1c) However, observations indicate that cell divisions are occurring all along an embryonic axis, such as a limb.[11, 16] Note, however, the affect that either rate changes in either cell divisions or gene expression, such as would result from even minor fluctuations in temperature, have on distance from the position versus time graph. If growth fluctuations occur, the relationships of cells to the gradient must also be altered in relationship to those positional changes and positions will vary with time.

During the initial embryonic stages most evidence seems to suggest that growth occurs with a sequential ordering of cells along the limb[11, 17] and body axis[18, 19], the chronological assignment of early positional values then may determine the final ordering of morphological structures.[11, 12, 20] A progress zone (PZ) model, was proposed in which an autonomous timing mechanism assigns positional values during the process of embryonic limb outgrowth.[12] Three requirements were proposed for this PZ timing mechanism: a positional value, a positional signal and some as yet unknown relationship of cell division to position. In support of the PZ hypothesis, each of a series sequentially arranged Hox genes was expressed in an ordinal manner along the limb axis, and each Hox gene product was first detected at the distal margin, possibly the PZ, of embryonic chick limbs [21] suggesting that cryptic Hox activation events occurred at the limb growth margins. These events might be interpreted to indicate that Hox proteins are directly involved in regulating gene expression during the initial stages of embryonic limb development. As indicated by equation 1.2, determining position during growth requires controlling cell divisions and gene expression in a simultaneous manner.

Similar to the PZ model, we came to understand a MRF model for the HOX code by applying our understanding of navigational methods, and methods used in engineering to analyze field events in conditions with velocity (FIG. 1d, see also FIG. 1b). Foremost, we sought to reduce the affects that changes in the growth rates might have on positional determinations, perhaps similar to the PZ conceptions. In engineering, the use of moving reference frame is well understood, MRFs are applied to the analysis of events with spatial field coordinates that occur near objects that are moving with constant velocity and function to keep time from appearing in the independent variable of governing equations. For example, consider the analysis of a moving heat source in a solid, in this case the temperature field is a function of both spatial coordinates and time.[22] By the introduction of a moving frame of velocity that coincides with that of the moving heat source, time is eliminated as a variable from the second order partial differential equation, the velocity of the heat source becomes a parameter in the differential equation written in terms that describe the moving frame and alters the determinants of the temperature field. With this scheme, time variations in the governing equation(s) to cell divisions and gene expression would be removed and the theoretical subordinate governing equation(s), regulating secondary and subsequent cell divisions and gene expression events, are determined by initial coordinates only, i.e., x, y, z.

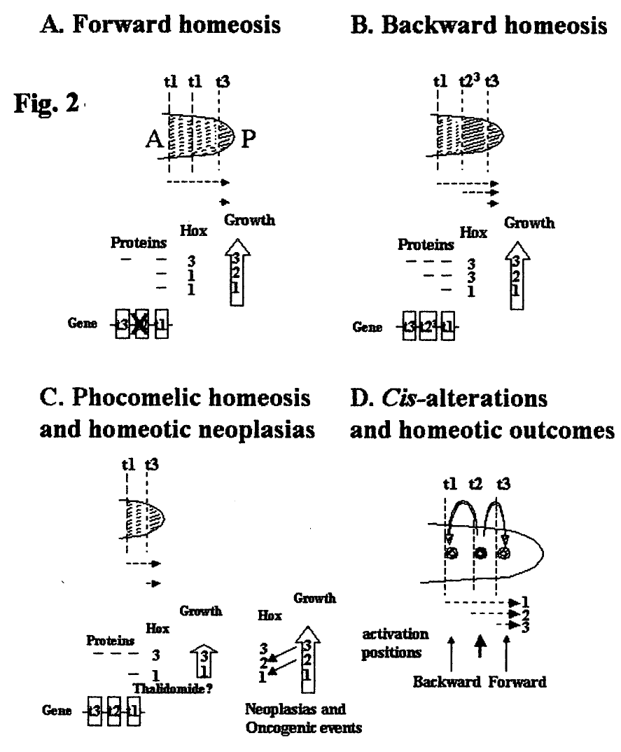

The importance of the initial position assignment by controlled cell divisions and gene expression, and role of position in defining subordinate decisions is shown diagrammatically as domain shifts that would result from mutations in Hox genes (FIG. 2a-c). One class of homeotic phenotypes, a backward homeosis, that suggests the reiteration of a morphological domain and might be expected to extend the total amount of axial growth allowed, when compared to a representative body plan, was specifically sought out in phenotypic variation studies of morphology.[5] Presently, allowing extensive additional cells along an axis such as is the case by forming or extending morphological domains to include cellular regions do not appear in a representative adult, suggests the occurrence of neoplastic events (FIG. 2c). Phocomelic dysmorphologies that can be described by developmental models,[11, 23] are anticipated by described homeotic variations.[5] A MRF model suggests that the antitumor effects of thalidomide in the treatment of multiple myeloma[24] could be caused by altering both the relationships gene expression and controlled cell divisions simultaneously (FIG. 2c).

Consider the application of MRF concepts and classical mechanics to the transformation of one segment identity to another, a homeosis. If we assign t₀=0 at the beginning of a morphological segment and apply two different MRFs, K and K^{f}, such as would be controlled by a normal and altered Hox proteins in the MRF model and suppose that the MRFS are coincident at t₀ and K^{f} is moving at constant velocity v greater than K, then the coordinate of an event in K at time t and position x are given in K^{f} by a Galilean transformation for changing over from one body of reference to another, equation (1.3). In this case, time relationships also hold, equation (1.4).
x^{f}=x−v(t₁−t₀) (1.3)
t^{f}=t (1.4)

Similar to the PZ model, we came to understand a MRF model for the HOX code by applying our understanding of navigational methods, and methods used in engineering to analyze field events in conditions with velocity (FIG. 1d, see also FIG. 1b). Foremost, we sought to reduce the affects that changes in the growth rates might have on positional determinations, perhaps similar to the PZ conceptions. In engineering, the use of moving reference frame is well understood, MRFs are applied to the analysis of events with spatial field coordinates that occur near objects that are moving with constant velocity and function to keep time from appearing in the independent variable of governing equations. For example, consider the analysis of a moving heat source in a solid, in this case the temperature field is a function of both spatial coordinates and time.[22] By the introduction of a moving frame of velocity that coincides with that of the moving heat source, time is eliminated as a variable from the second order partial differential equation, the velocity of the heat source becomes a parameter in the differential equation written in terms that describe the moving frame and alters the determinants of the temperature field. With this scheme, time variations in the governing equation (s) to cell divisions and gene expression would be removed and the theoretical subordinate governing equation(s), regulating secondary and subsequent cell divisions and gene expression events, are determined by initial coordinates only, i.e., x, y, z.

The importance of the initial position assignment by controlled cell divisions and gene expression, and role of position in defining subordinate decisions is shown diagrammatically as domain shifts that would result from mutations in Hox genes (FIG. 2a-c). One class of homeotic phenotypes, a backward homeosis, that suggests the reiteration of a morphological domain and might be expected to extend the total amount of axial growth allowed, when compared to a representative body plan, was specifically sought out in phenotypic variation studies of morphology.[5] Presently, allowing extensive additional cells along an axis such as is the case by forming or extending morphological domains to include cellular regions do not appear in a representative adult, suggests the occurrence of neoplastic events (FIG. 2c). Phocomelic dysmorphologies that can be described by developmental models,[1, 23] are anticipated by described homeotic variations.[5] A MRF model suggests that the antitumor effects of thalidomide in the treatment of multiple myeloma[24] could be caused by altering both the relationships gene expression and controlled cell divisions simultaneously (FIG. 2c).

The growth-synchronizing biological MRF discussed here seems different from MRFs used in classical mechanics. First, the velocity of the biological MRF is not set by the outside observer to coincide with the internal reference frame, instead the control occurs by assigning cell divisions and regulating gene expression in synchrony at the MRF, in essence producing spatial-temporal biological time (FIG. 2d) as suggested by dock models for development. The moving reference frame, in part, may cause these homeotic outcomes and alterations or mutations in DNA elements (that are the target of the HOX code MRF regulatory system) can be used to alter positioning of organs or features in a homeotic manner.[5]

The redundant involvement of Hox proteins, such as HoxD13, in the formation of different morphological axes, for example the genitalia and the limb,[25] suggests that distance, s, can be altered by changing the interpreted value of t, by altering the cell divisions and gene expression in synchrony. As well, specific but differing series of Hox proteins may be involved in the formation of each of the many morphological axes. This capacity to regulate biological time t to control axis distance, s, using a specific series of Hox proteins suggests that the correct terminology for this process of position determination in biological systems is time-varying growth-synchronizing MRF. This time-varying nomenclature should not be confused, however, with time-varying reference frames that function with 24 hour dock.

Visualization of the control of position as an observer stationed near the tip and traveling at the speed of growth in this initial step greatly enhances our understanding of the complicated process of growth along an axis. The introduction of MRF concepts with laboratory observations may be a crucial step toward developing theoretical descriptions of governing equations for cell divisions and gene expression during the initial growth stages, extension of a metazoan morphological axis. This MRF model terminology, as opposed to the PZ, better reflects this biological capacity to replicate almost identically spatial and temporally related morphological structures, at different velocities, and different times, generation after generation.
1. Gaunt, S. J., et al., Homoeobox gene expression in mouse embryos varies with position by the primitive streak stage. Nature, 1986. 324(6098): p. 662-4.
2. Dolle, P., et al., Coordinate expression of the murine Hox-5 complex homoeobox-containing genes during limb pattern formation. Nature, 1989. 342(6251): p. 767-72.
3. Sauvageau, G., et al., Overexpression of HOXB4 in hematopoietic cells causes the selective expansion of more primitive populations in vitro and in vivo. Genes Dev, 1995. 9(14): p. 1753-65.
4. Antonchuk, J., G. Sauvageau, and R. K. Humphries, HOXB4-induced expansion of adult hematopoietic stem cells ex vivo. Cell, 2002. 109(1): p. 39-45.
5. Bateson, W., Materials for the study of variation treated with especial regard for the discontinuity in the origin of the species. 1894, Cambridge: University Press.
6. Carrolll, S. B., Homeotic genes and the evolution of arthropods and chordates. Nature, 1995. 376(6540): p. 479-85.
7. Slack, J. M., P. W. Holland, and C. F. Graham, The zootype and the phylotypic stage. Nature, 1993. 361(6412): p. 490-2.
8. Lewis, E. B., A gene complex controlling segmentation in Drosophila. Nature, 1978. 276 (5688): p. 565-70.
9. Kessel, M. and P. Gruss, Homeotic transformations of murine vertebrae and concomitant alteration of Hox codes induced by retinoic acid. Cell, 1991. 67(1): p. 89-104.
10. Lufkin, T., et al., Disruption of the Hox-1.6 homeobox gene results in defects in a region corresponding to its rostral domain of expression. Cell, 1991. 66(6): p. 1105-19.
11. Saunders, J. W., Jr., The proximo-distal sequence of origin of the parts of the chick wing and the role of the ectoderm. J Exp Zool, 1948. 108: p. 363-403.
12. Summerbell, D., J. H. Lewis, and L. Wolpert, Positional information in chick limb morphogenesis. Nature, 1973. 244(5417): p. 492-6.
13. Duboule, D., Making progress with limb models. Nature, 2002. 418(6897): p. 492-3.
14. Saunders, J. W., Jr., is the progress zone model a victim of progress? Cell, 2002. 110 (5): p. 541-3.
15. Tickle, C. and L. Wolpert, The progress zone—alive or dead? Nat Cell Biol, 2002. 4(9): p. E216-7.

16. Hormbruch, A. and L. Wolpert, Cell division in the early growth and morphogenesis of the chick limb. Nature, 1970. 226(247): p. 764-6.
17. Vargesson, N., et al., Cell fate in the chick limb bud and relationship to gene expression. Development, 1997.124(10): p. 1909-18.

18. Stern, C. D., Mesoderm induction and development of the embryonic axis in amniotes. Trends Genet, 1992. 8(5): p. 158-63.
19. Nicolas, J. F., et al., Evidence in the mouse for self-renewing stem cells in the formation of a segmented longitudinal structure, the myotome. Development, 1996. 122(9): p. 293346.
20. Dubrulle, J., M. J. McGrew, and O. Pourquie, FGF signaling controls somite boundary position and regulates segmentation clock control of spatiotemporal Hox gene activation. Cell, 2001. 106(2): p. 219-32.
21. Izpisua-Belmonte, J. C., et al., Expression of the homeobox Hox-4 genes and the specification of position in chick wing development. Nature, 1991. 350(6319): p. 585-9.
22. Rosenthal, D., The theory of moving sources of heat and its application to metal treatments. Trans ASME, 1946. 68: p. 849-866.
23. Tabin, C. J., A developmental model for thalidomide defects. Nature, 1998. 396(6709): p. 322-3.
24. Singhal, S., et al., Antitumor activity of thalidomide in refractory multiple myeloma. N Engl J Med, 1999. 341(21): p. 1565-71.
25. Dolle, P., et al., HOX-4 genes and the morphogenesis of mammalian genitalia. Genes Dev, 1991. 5(10): p. 1767-7.
26. Crick, F., Diffusion in embryogenesis. Nature, 1970. 5231(231): p. 420-2.
27. Pfeiffer, R. A. a. W. K, Thalidomide and congenital abnormalities. Lancet, 1962: p. 46-47.
28. Lenz, W., Thalidomide and congenital abnormalities. Lancet, 1962: p. 46-47.

Program Listing Deposit

[insert program here]

1. In this invention we describe methods to produce organisms at different geological, or sometimes termed absolute times, and explain fundamental solutions to the space-time relationships of metazoans organisms to one another.

2. We describe fundamental functions of the HOX code and its role in carcinogenesis.

3. We describe the fundamental underlying methods for theoretical descriptions of cellular positions and the relationship of normal growth and cellular positions to cancers and neoplasias. Such that cancer is associated with the methods required for the determination of position during the growth of metazoan organisms. If position is not maintained as a direct relationship to cell division and/or gene expression, then time will appear as an independent variable of a theoretical governing equations that describe growth and the positions.

4. Organs and morphological features can be repositioned into differing segments by alternating DNA elements that are the target of the HOX code, in part due to this moving reference system.
